(19) United States
(12) Patent Application Publication
(10) Pub. No.: US 2018/0340054 A1
(43) Pub. Date: Nov. 29, 2018

(54) TRI-ESTERS OF 1,2,4-BENZENETRIOL AS PLASTICISERS IN THE POLYMERIC COMPOSITIONS

(71) Applicant: OIL REFINERIES LTD., Haifa (IL)

(72) Inventor: Avraham Meshulam, Hadera (IL)

(21) Appl. No.: 15/534,012

(22) PCT Filed: Nov. 29, 2015
(86) PCT No.: PCT/IL2015/051159

§ 371 Date: Jun. 8, 2017

(30) Foreign Application Priority Data
Dec. 8, 2014 (IL) 236137

(51) Int. Cl. C08K 5/134

(52) U.S. Cl. CPC C08K 5/134; C08K 5/1345

(57) ABSTRACT

The invention provides plasticisers for polymeric compositions, particularly advantageous for PVC compositions, exhibiting a good heat and mechanical stability, as well as a low toxicity. The plasticisers are tri-esters of 1,2,4-benzenetriol and can be obtained in a low-cost process.

### FIELD OF THE INVENTION

The present invention relates to the field of polymeric and polyvinyl chloride compositions in general, and to new plasticisers for the polyvinyl chloride compositions, in particular.

### BACKGROUND OF THE INVENTION

Plasticisers are colourless, odourless, organic chemicals used to soften polyvinyl chloride (PVC) and other polymers creating a whole new world of soft and bendable polymers for high performing applications and uses, which bring a myriad of benefits to everyday life. These include a vast variety of durable goods, which can be used in the construction, transport and telecommunication.

Today, the majority of the plasticisers are widely employed in flexible PVC applications, largely for the construction, automotive and wire and cable sectors. In fact, plasticisers cannot be simply treated as additives like pigments or fillers. They are the major functional additives that determine and improve the physical properties of PVC and other polymers.

Typically, these plasticisers are esters of polycarboxylic acids with linear or branched aliphatic alcohols of moderate chain length that have low vapour pressure and good heat stability. Most of them are chemically inert and compatible with polyvinyl chloride and other host materials.

Ester plasticizers are selected based upon cost-performance evaluation for their compatibility, processability, permanence, volatility and other performance properties. The examples of the ester plasticisers that are used in the PVC and other plastics industries include adipates, sebacates, dibenzoates, gluterates, trimellitates, maleates, azelates, terephthalates and phthalates.

Various phthalate esters of straight-chain and branched-chain alkyl alcohols obtained from a reaction between phthalic acid and the corresponding alcohol are the most common type of general plasticisers and form a diverse family of chemical substances. They are generally non-toxic, meet the cost-performance requirements and have traditionally been the most dominant plasticisers. However, the recent regulatory concerns have led to pressure to change to non-phthalate plasticisers, especially in Europe. This is because phthalate esters are relatively low molecular weight compounds that interact with polyvinyl chloride through physical interactions. As a result of their low molecular weight and the lack of stable chemical bonds between them and the PVC chains, phthalate esters can easily be extracted from the polymer matrix. Traces or higher amounts have been detected in most parts of the environment, in animals and in humans.

When plastic materials are used in applications such as medical devices or toys, they come in contact with biological fluids. Biological fluids such as gastric juice or saliva provide an aggressive environment that accelerates the release of low molecular weight additives. Exudation of plasticisers changes the long-term properties of the material and may pose a threat to animals and humans if the plasticiser is passed on to them.

Phthalate plasticisers have, in in vitro and toxicological animal tests, been shown to have adverse effects in the liver, the reproductive tract, the kidneys, the lungs, the heart, and on the fetus. Phthalate esters have been identified as irritants and immunogens of respiratory syndromes, and toxicological data, together with the limited human-exposure data, leads to a concern that bis(2-ethylhexyl)phthalate (DEHP), one of the most common phthalate plasticisers used in medical devices, is harmful to human fertility and reproduction. DEHP is also a suspected endocrine disruptor and there are indications that MEHP, the principal hydrolysis product of DEHP, exhibits genotoxic effects in human mucosal cells and lymphocytes.

Much safer plasticisers with better biodegradability and fewer biological effects, such as citrates, acetylated monoglycerides and epoxidized vegetable oils have been recently developed. However, their thermal stability is relatively low and their production cost is relatively high.

Apart from phthalates, several other kinds of plasticisers are used to meet specific requirements, including adipates for low temperature resistance and trimellitates for heat resistance.

Trimellitate plasticisers obtained by the reaction of trimellitic anhydride with aliphatic alcohols are primary non-phthalate plasticisers for polyvinyl chloride resins and copolymers. They give the plasticised PVC good workability, a good resistance to high temperatures with retention of the mechanical properties, a high resistance to migration and extraction in aqueous solutions, and good flexibility at low temperatures.

Trimellitates, such as tris-(2-ethylhexyl) trimellitate (TOTM), have very low volatility, low fogging, high permanence for increased service life, and are typically used in high-specification electrical cable insulation and sheathing, usually in blends with the highest-molecular-weight phthalates. However, their manufacturing cost is extremely high because of the high cost of their production facilities made from titanium. In addition to high cost of this manufacturing process, there is a severe ecological impact related to production of trimellitic anhydride, which is an intermediate in the synthesis of trimellitic acid.

In general, the manufacturing process for trimellitic acid from pseudocumene (1,2,4-trimethyl benzene), is described in U.S. Pat. No. 4,948,921. The process involves a catalytic liquid phase oxidation of commercially available pseudocumene with air or bromine/bromic acid in the presence of acetic acid as a reaction solvent, separation and recovery of crystalline trimellitic acid, followed by thermal dehydration of trimellitic acid to its anhydride and hydrolysing the trimellitic anhydride.

The manufacturing process of the present invention overcomes the aforementioned problems by preparing a raw material via Mid-Century oxidation process, whereas the recovery and recycle of acetic acid and recovery of methyl acetate are essentially as practiced by dimethyl terephthalate technology, which appears to be one of the lowest polluters, and its predicted pre-eminence will suppress future emissions totals.

Thus, for a successful replacement of the aforementioned plasticisers, the new material must not only provide the necessary technical properties but must also be processable in ecologically safe conventional processing equipment at a comparable cost. The demand for new alternative plasticisers is thus still strong, and relatively cheap and non-toxic plasticisers are one of the most appealing solutions.

### SUMMARY OF THE INVENTION

The present invention relates to a group of new plasticisers for the polyvinyl chloride (PVC) compositions. In particular, these new plasticisers are tri-esters of 1,2,4-benzenetriol, which demonstrate good resistance to high temperatures with retention of the mechanical properties, and are very cheap in production.

A first aspect of the present invention is a polymeric composition comprising a blend of a polymer and one or more compounds of Formula (I):

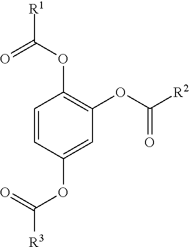

wherein R¹, R² and R³ independently represent C₁-C₁₈ alkyl; C₂-C₁₈ alkenyl; C₂-C₁₈ alkynyl; C₃-C₇ cycloalkyl; C₃-C₇ cycloalkenyl; C₃-C₇ cycloalkyl-C₁-C₁₈ alkyl; C₁-C₁₈ haloalkyl; C₂-C₁₈ haloalkenyl; C₂-C₁₈ haloalkynyl; C₃-C₇ halocycloalkyl; C₃-C₇ halocycloalkyl-C₁-C₁₈ alkyl; amino-C₁-C₁₈ alkyl; nitro-C₁-C₁₈ alkyl; C₁-C₁₈ alkylamino-C₁-C₁₈ alkyl; di-(C₁-C₁₈ alkyl)amino-C₁-C₁₈ alkyl; C₁-C₁₈ alkylthio-C₁-C₁₈ alkyl; C₁-C₁₈ alkylsulfinyl-C₁-C₁₈ alkyl; C₁-C₁₈ alkylsulfonyl-C₁-C₁₈ alkyl; C₁-C₁₈ haloalkylthio-C₁-C₁₈ alkyl; C₁-C₁₈ haloalkylsulfinyl-C₁-C₁₈ alkyl; C₁-C₁₈ haloalkylsulfonyl-C₁-C₁₈ alkyl; C₁-C₁₈ alkoxy-C₁-C₁₈ alkyl; hydroxy-C₁-C₁₈ alkyl; aryl-C₁-C₁₈ alkoxy-C₁-C₁₈ alkyl; C₂-C₂₄ polyethylene oxide; C₃-C₇ cycloalkyloxy-C₁-C₁₈ alkyl; C₃-C₇ cycloalkyl-C₁-C₁₈ alkyloxy-C₁-C₁₈ alkyl; aryloxy-C₁-C₁₈ alkyl; arylthio-C₁-C₁₈ alkyl; arylsulfinyl-C₁-C₁₈ alkyl; arylsulfonyl-C₁-C₁₈ alkyl; C₁-C₁₈ haloalkoxy-C₁-C₁₈ alkyl; aryl-C₁-C₁₈ alkyl; aryl-C₂-C₁₈ alkenyl; aryl-C₂-C₁₈ alkynyl; C₁-C₁₈ alkoxyimino-C₁-C₁₈ alkyl; aryloxyimino-C₁-C₁₈ alkyl; di-(C₁-C₁₈ alkoxy)C₁-C₁₈ alkyl; (R⁴R⁵N—C═O)C₁-C₁₈ alkyl; C₁-C₁₈ alkoxy-carbonyl C₁-C₁₈ alkyl; C₁-C₁₈ alkylcarbonyl-C₁-C₁₈ alkyl; C₁-C₁₈ alkyl-carbonyloxy-C₁-C₁₈ alkyl; C₁-C₁₈ alkylidene aminooxy-C₁-C₁₈ alkyl; formyl C₁-C₁₈ alkyl; C₁-C₁₈ alkylthio-C₁-C₁₈ alkoxy-C₁-C₁₈ alkyl; C₁-C₁₈ alkylsulfinyl-C₁-C₁₈ alkoxy-C₁-C₁₈ alkyl; C₁-C₁₈ alkylsulfonyl-C₁-C₁₈ alkoxy-C₁-C₁₈ alkyl; cyano C₁-C₁₈ alkyl; cyano-C₁-C₁₈ alkoxy-C₁-C₁₈ alkyl; C₂-C₁₈ alkylidene amino; di-(C₁-C₁₈ alkyl)amino-C₁-C₁₈ alkylidene amino; NR⁴R⁵; aryl; heterocyclyl comprising 1 to 10 carbon atoms and one or more same or different heteroatoms selected from oxygen, sulfur optionally oxidized to sulfoxide or sulfone, and nitrogen; C₁-C₁₈ alkyl group substituted with heterocyclyl comprising 1 to 10 carbon atoms and one or more same or different heteroatoms selected from oxygen, sulfur optionally oxidized to sulfoxide or sulfone, and nitrogen; C₁-C₁₈ alkoxy-C₁-C₁₈ alkyl group substituted with heterocyclyl comprising 1 to 10 carbon atoms and one or more same or different heteroatoms selected from oxygen, sulfur optionally oxidized to sulfoxide or sulfone, and nitrogen;

R⁴ and R⁵ each independently represent a hydrogen atom; C₁-C₁₈ alkyl; aryl; C₁-C₁₈ alkoxy-C₁-C₁₈ alkyl; C₁-C₁₈ alkylcarbonyl; C₁-C₁₈ alkylthio carbonyl; C₁-C₁₈ alkoxycarbonyl; C₁-C₁₈ haloalkyl; C₃-C₇ cycloalkyl; C₃-C₇ cycloalkyl-C₁-C₁₈ alkyl; C₁-C₁₈ alkylsulfonyl; and arylsulfonyl;

R⁴ and R⁵ may be joined together to form a 5- to 6-membered ring with adjacent nitrogen atom, and the one or more carbon atoms in the ring may be substituted with a sulfur atom and an oxygen atom;

wherein aryl and heterocyclyl can be substituted with 0 to 5 same or different substituents selected from a halogen atom; a hydroxyl group; C₁-C₁₈ alkyl; C₃-C₇ cycloalkyl; C₃-C₇ cycloalkyl-C₁-C₁₈ alkyl; C₂-C₁₈ alkenyl; C₂-C₁₈ alkynyl; C₁-C₁₈ haloalkyl; C₂-C₁₈ haloalkenyl; C₂-C₁₈ haloalkynyl; C₃-C₇ halocycloalkyl; C₃-C₇ halocycloalkyl-C₁-C₁₈ alkyl; C₁-C₁₈ alkoxy; C₃-C₇ cycloalkyloxy; C₂-C₁₈ alkenyloxy; C₂-C₁₈ alkynyloxy; C₁-C₁₈ alkylcarbonyloxy; C₁-C₁₈ haloalkoxy; a C₁-C₁₈ alkylthio; a C₁-C₁₈ alkylsulfinyl; a C₁-C₁₈ alkylsulfonyl; a C₁-C₁₈ haloalkylthio; a C₁-C₁₈ haloalkylsulfinyl; a C₁-C₁₈ haloalkylsulfonyl; an amino group; C₁-C₁₈ alkylcarbonylamino; mono(C₁-C₁₈ alkyl)amino; di-(C₁-C₁₈ alkyl)amino; hydroxy-C₁-C₁₈ alkyl; C₁-C₁₈ alkoxy-C₁-C₁₈ alkyl; C₁-C₁₈ alkylthio-C₁-C₁₈ alkyl; C₁-C₁₈ alkylsulfinyl-C₁-C₁₈ alkyl; C₁-C₁₈ alkylsulfonyl-C₁-C₁₈ alkyl; C₁-C₁₈ haloalkylthio-C₁-C₁₈ alkyl; C₁-C₁₈ haloalkylsulfinyl-C₁-C₁₈ alkyl; C₁-C₁₈ haloalkylsulfonyl-C₁-C₁₈ alkyl; cyano-C₁-C₁₈ alkyl; C₁-C₁₈ alkoxy-C₁-C₁₈ alkoxy; C₃-C₇ cycloalkyl-C₁-C₁₈ alkyloxy; C₁-C₁₈ haloalkoxy-C₁-C₁₈ alkoxy; cyano-C₁-C₁₈ alkoxy; C₁-C₁₈ acyl; C₁-C₁₈ alkoxyimino-C₁-C₁₈ alkyl; a carboxylic acid group; C₁-C₁₈ alkoxycarbonyl; carbamoyl; nitro; cyano; aryl; sulfonyl, sulfinyl, formyl, thiol, isocyanate or isothiocyanate mono(C₁-C₁₈ alkyl)aminocarbonyl; di-(C₁-C₁₈ alkyl)amino-carbonyl; and C₃-C₇ alkylene group formed with two adjacent substituent groups, wherein 1 to 3 carbon atoms in the alkylene group may be substituted with an atom selected from a group consisting of an oxygen atom, a sulfur atom, a nitrogen atom, and a carbon atom constituting a carbonyl.

Another aspect of the present invention relates to use of the compound of Formula (I) as plasticiser in the polymeric composition. According to a particular aspect of the present invention, the polymer is polyvinyl chloride (PVC).

A further aspect of the present invention is a preparation process of the compound of Formula (I) comprising the one-step esterification reaction between one equivalent of 1,2,4-benzenetriol and at least 3.5 times excess of a carboxylic acid.

Various embodiments of the invention may allow various benefits, and may be used in conjunction with various applications. The details of one or more embodiments are set forth in the accompanying figures and the description below. Other features, objects and advantages of the described techniques will be apparent from the description and drawings and from the claims.

The present invention will be understood and appreciated more fully from the following detailed description taken in conjunction with the reaction scheme showing the chemical reaction used in the preparation process of the compound of Formula (I).

### DETAILED DESCRIPTION OF THE INVENTION

In the following description, various aspects of the invention will be described. For purposes of explanation, specific configurations and details are set forth in order to provide a thorough understanding of the invention. However, it will also be apparent to one skilled in the art that the invention may be practiced without the specific details presented herein. Furthermore, well-known features may be omitted or simplified in order not to obscure the invention.

It should be noted that although a portion of the discussion may relate to the polymeric compositions comprising a blend of a polymer and one or more new plasticisers, the present invention is not limited in this regard, and embodiments of the present invention may be used in conjunction with various other compositions and processes. As such, some embodiments of the invention may be used, for example, in conjunction with automobile, textile or plastic industry or in any other related applications or processes.

The present invention is a polymeric composition comprising a blend of a polymer and one or more compounds of Formula (I):

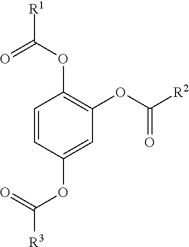

wherein R¹, R² and R³ independently represent C₁-C₁₈ alkyl; C₂-C₁₈ alkenyl; C₂-C₁₈ alkynyl; C₃-C₇ cycloalkyl; C₃-C₇ cycloalkenyl; C₃-C₇ cycloalkyl-C₁-C₁₈ alkyl; C₁-C₁₈ haloalkyl; C₂-C₁₈ haloalkenyl; C₂-C₁₈ haloalkynyl; C₃-C₇ halocycloalkyl; C₃-C₇ halocycloalkyl-C₁-C₁₈ alkyl; amino-C₁-C₁₈ alkyl; nitro-C₁-C₁₈ alkyl; C₁-C₁₈ alkylamino-C₁-C₁₈ alkyl; di-(C₁-C₁₈ alkyl)amino-C₁-C₁₈ alkyl; C₁-C₁₈ alkylthio-C₁-C₁₈ alkyl; C₁-C₁₈ alkylsulfinyl-C₁-C₁₈ alkyl; C₁-C₁₈ alkylsulfonyl-C₁-C₁₈ alkyl; C₁-C₁₈ haloalkylthio-C₁-C₁₈ alkyl; C₁-C₁₈ haloalkylsulfinyl-C₁-C₁₈ alkyl; C₁-C₁₈ haloalkylsulfonyl-C₁-C₁₈ alkyl; C₁-C₁₈ alkoxy-C₁-C₁₈ alkyl; hydroxy-C₁-C₁₈ alkyl; aryl-C₁-C₁₈ alkoxy-C₁-C₁₈ alkyl; C₂-C₂₄ polyethylene oxide; C₃-C₇ cycloalkyloxy-C₁-C₁₈ alkyl; C₃-C₇ cycloalkyl-C₁-C₁₈ alkyloxy-C₁-C₁₈ alkyl; aryloxy-C₁-C₁₈ alkyl; arylthio-C₁-C₁₈ alkyl; arylsulfinyl-C₁-C₁₈ alkyl; arylsulfonyl-C₁-C₁₈ alkyl; C₁-C₁₈ haloalkoxy-C₁-C₁₈ alkyl; aryl-C₁-C₁₈ alkyl; aryl-C₂-C₁₈ alkenyl; aryl-C₂-C₁₈ alkynyl; C₁-C₁₈ alkoxyimino-C₁-C₁₈ alkyl; aryloxyimino-C₁-C₁₈ alkyl; di-(C₁-C₁₈ alkoxy)C₁-C₁₈ alkyl; (R⁴R⁵N—C═O)C₁-C₁₈ alkyl; C₁-C₁₈ alkoxycarbonyl C₁-C₁₈ alkyl; C₁-C₁₈ alkylcarbonyl-C₁-C₁₈ alkyl; C₁-C₁₈ alkyl-carbonyloxy-C₁-C₁₈ alkyl; C₁-C₁₈ alkylidene aminooxy-C₁-C₁₈ alkyl; formyl-C₁-C₁₈ alkyl; C₁-C₁₈ alkylthio-C₁-C₁₈ alkoxy-C₁-C₁₈ alkyl; C₁-C₁₈ alkylsulfinyl-C₁-C₁₈ alkoxy-C₁-C₁₈ alkyl; C₁-C₁₈ alkylsulfonyl-C₁-C₁₈ alkoxy-C₁-C₁₈ alkyl; cyano-C₁-C₁₈ alkyl; cyano-C₁-C₁₈ alkoxy-C₁-C₁₈ alkyl; C₂-C₁₈ alkylidene amino; di-(C₁-C₁₈ alkyl)amino-C₁-C₁₈ alkylidene amino; NR⁴R⁵; aryl; heterocyclyl comprising 1 to 10 carbon atoms and one or more same or different heteroatoms selected from oxygen, sulfur optionally oxidized to sulfoxide or sulfone, and nitrogen; C₁-C₁₈ alkyl group substituted with heterocyclyl comprising 1 to 10 carbon atoms and one or more same or different heteroatoms selected from oxygen, sulfur optionally oxidized to sulfoxide or sulfone, and nitrogen; C₁-C₁₈ alkoxy-C₁-C₁₈ alkyl group substituted with heterocyclyl comprising 1 to 10 carbon atoms and one or more same or different heteroatoms selected from oxygen, sulfur optionally oxidized to sulfoxide or sulfone, and nitrogen;

R⁴ and R⁵ each independently represent a hydrogen atom; C₁-C₁₈ alkyl; aryl; C₁-C₁₈ alkoxy-C₁-C₁₈ alkyl; C₁-C₁₈ alkylcarbonyl; C₁-C₁₈ alkylthio carbonyl; C₁-C₁₈ alkoxycarbonyl; C₁-C₁₈ haloalkyl; C₃-C₇ cycloalkyl; C₃-C₇ cycloalkyl-C₁-C₁₈ alkyl; C₁-C₁₈ alkylsulfonyl; and arylsulfonyl;

R⁴ and R⁵ may be joined together to form a 5- to 6-membered ring with adjacent nitrogen atom, and the one or more carbon atoms in the ring may be substituted with a sulfur atom and an oxygen atom;

wherein aryl and heterocyclyl are substituted with 0 to 5 same or different substituents selected from a halogen atom; a hydroxyl group; C₁-C₁₈ alkyl; C₃-C₇ cycloalkyl; C₃-C₇ cycloalkyl-C₁-C₁₈ alkyl; C₂-C₁₈ alkenyl; C₂-C₁₈ alkynyl; C₁-C₁₈ haloalkyl; C₂-C₁₈ haloalkenyl; C₂-C₁₈ haloalkynyl; C₃-C₇ halocycloalkyl; C₃-C₇ halocycloalkyl-C₁-C₁₈ alkyl; C₁-C₁₈ alkoxy; C₃-C₇ cycloalkyloxy; C₂-C₁₈ alkenyloxy; C₂-C₁₈ alkynyloxy; C₁-C₁₈ alkylcarbonyloxy; C₁-C₁₈ haloalkoxy; a C₁-C₁₈ alkylthio; a C₁-C₁₈ alkylsulfinyl; a C₁-C₁₈ alkylsulfonyl; a C₁-C₁₈ haloalkylthio; a C₁-C₁₈ haloalkylsulfinyl; a C₁-C₁₈ haloalkylsulfonyl; an amino group; C₁-C₁₈ alkylcarbonylamino; mono(C₁-C₁₈ alkyl)amino; di-(C₁-C₁₈ alkyl)amino; hydroxy-C₁-C₁₈ alkyl; C₁-C₁₈ alkoxy-C₁-C₁₈ alkyl; C₁-C₁₈ alkylthio-C₁-C₁₈ alkyl; C₁-C₁₈ alkylsulfinyl-C₁-C₁₈ alkyl; C₁-C₁₈ alkylsulfonyl-C₁-C₁₈ alkyl; C₁-C₁₈ haloalkylthio-C₁-C₁₈ alkyl; C₁-C₁₈ haloalkylsulfinyl-C₁-C₁₈ alkyl; C₁-C₁₈ haloalkylsulfonyl-C₁-C₁₈ alkyl; cyano-C₁-C₁₈ alkyl; C₁-C₁₈ alkoxy-C₁-C₁₈ alkoxy; C₃-C₇ cycloalkyl-C₁-C₁₈ alkyloxy; C₁-C₁₈ haloalkoxy-C₁-C₁₈ alkoxy; cyano-C₁-C₁₈ alkoxy; C₁-C₁₈ acyl; C₁-C₁₈ alkoxyimino-C₁-C₁₈ alkyl; a carboxylic acid group; C₁-C₁₈ alkoxycarbonyl; carbamoyl; nitro; cyano; aryl; sulfonyl, sulfinyl, formyl, thiol, isocyanate or isothiocyanate mono(C₁-C₁₈ alkyl)aminocarbonyl; di-(C₁-C₁₈ alkyl)amino-carbonyl; and C₃-C₇ alkylene group formed with two adjacent substituent groups, wherein 1 to 3 carbon atoms in the alkylene group may be substituted with an atom selected from a group consisting of an oxygen atom, a sulfur atom, a nitrogen atom, and a carbon atom constituting a carbonyl.

The term “alkyl” refers to a linear or branched chain saturated monovalent hydrocarbon radical. Exemplary alkyl groups include methyl, ethyl, propyl, isopropyl, 1-methylpropyl, n-butyl, tert-butyl, isobutyl, pentyl, isopentyl, hexyl, isohexyl, heptyl, dimethylpentyl, diethylpentyl, octyl, nonyl, decyl, undecyl, dodecyl, 2,2,4-trimethylpentyl and the like. The term “C₁-C₁₈ alkyl” refers to a linear or branched chain alkyl containing from 1 to 18 carbon atoms. When alkyl is used as a suffix following another named group, such as “haloalkyl” or “hydroxy-C₁-C₁₈”, this is intended to refer to an alkyl having bonded thereto one, two or three of other, specifically-named groups, such as halogen or hydroxy, at any point of attachment on either the straight or branched chain of the alkyl.

The term “alkenyl” refers to a linear or branched chain unsaturated divalent (—CH═CH—) hydrocarbon radical. Exemplary alkenyl groups are ethylene, propylene isomers and the like. The term “alkynyl” refers to an unsaturated trivalent (—C≡C—) (linear) hydrocarbon radical, such as acetylene, propyne, butyne isomers and the like.

The term “aryl” refers to a monovalent unsaturated aromatic hydrocarbon radical of 6 to 18 ring atoms having a single ring or multiple condensed rings. Exemplary aryl groups are phenyl, biphenyl, naphthyl, anthryl, pyrenyl and the like. When the term “substituted” is used with such groups, as in “substituted with 0 to 5 same or different substituents”, it should be understood that the aryl moiety may be substituted with the groups selected from those recited above and hereinafter as appropriate.

The term “cycloalkyl” refers to a fully saturated and partially unsaturated cyclic monovalent hydrocarbon radical of 1 to 4 rings and 3 to 8 carbons per ring. Exemplary cycloalkyl groups are cyclopropyl, cyclobutyl, cyclopentyl, cyclobutenyl, cyclopentenyl, cyclohexenyl, adamantyl and the like. A cycloalkyl ring may have a carbon ring atom replaced with a carbonyl group (C═O). The cycloalkyl ring can be fused to aryl or heteroaryl ring. Cycloalkyl groups include such rings having a second or third ring fused thereto that is a heterocyclo, heteroaryl, or aryl group, provided that in such cases the point of attachment is to the cycloalkyl portion of the ring system. The term “cycloalkyl” also includes such rings having a second or third ring attached to the ring or ring system in a Spiro fashion wherein the spiro ring is either a heterocyclo or carbocyclic ring.

The terms “heterocyclic” and “heterocyclyl” refer to fully saturated or partially unsaturated non-aromatic cyclic radicals of 3 to 8 ring atoms in each cycle (3 to 8 atoms in a monocyclic group, 6 to 12 atoms in a bicyclic group, and 10 to 18 atoms in a tricyclic group), which have at least one heteroatom (nitrogen, oxygen or sulphur) and at least one carbon atom in a ring. Each ring of the heterocyclic group containing a heteroatom may have from 1 to 4 heteroatoms, where the nitrogen and/or sulphur heteroatoms may optionally be oxidised and the nitrogen heteroatoms may optionally be quaternised. A heterocyclyl group may have a carbon ring atom replaced with a carbonyl group (—C═O). The heterocyclyl group may be attached to the remainder of the molecule at any nitrogen atom or carbon atom of the ring or ring system. Additionally, the heterocyclo group may have a second or third ring attached thereto in a Spiro or fused fashion, provided the point of attachment is to the heterocyclyl group. An attached spiro ring may be a carbocyclic or heterocyclic ring and the second and/or third fused ring may be a cycloalkyl, aryl or heteroaryl ring. Exemplary monocyclic heterocyclic groups include azetidinyl, oxiranyl, pyrrolidinyl, pyrazolinyl, imidazolidinyl, dioxanyl, dioxolanyl, oxazolidinyl, piperidinyl, piperazinyl, morpholinyl, tetrahydrofuryl, tetrahydropyranyl, thiamorpholinyl, and the like. Exemplary bicyclic heterocyclic groups include indolinyl, isoindolinyl, quinuclidinyl, benzopyrrolidinyl, benzopyrazolinyl, benzoimidazolidinyl, benzopiperidinyl, tetrahydroisoquinolinyl, tetrahydroquinolinyl, dihydroisoindolyl, and the like.

The term “heteroaryl” refers to aromatic monocyclic, bicyclic or tricyclic radicals of 3 to 8 ring atoms in each cycle (for example, 3 to 8 atoms in a monocyclic group, 6 to 12 atoms in a bicyclic group, and 10 to 18 atoms in a tricyclic group), which have at least one heteroatom (nitrogen, oxygen or sulphur) and at least one carbon atom in a ring. Each ring of the heteroaryl group may have 1-4 heteroatoms, wherein nitrogen and/or sulphur may optionally be oxidised, and the nitrogen heteroatoms may optionally be quaternised. The heteroaryl group may be attached to the remainder of the molecule at any nitrogen atom or carbon atom of the ring or ring system. Additionally, the heteroaryl group may have a second or third carbocyclic (cycloalkyl or aryl) or heterocyclic ring fused thereto provided the point of attachment is to the heteroaryl group. Exemplary “heteroaryl” groups are pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and so on. Exemplary bicyclic heteroaryl groups include benzothiazolyl, benzoxazolyl, quinolinyl, benzoxadiazolyl, benzothienyl, chromenyl, indolyl, indazolyl, isoquinolinyl, benzimidazolyl, benzopyranyl, benzofuryl, benzofurazanyl, benzopyranyl, cinnolinyl, quinoxalinyl, pyrrolopyridyl, furopyridinyl (such as furo[2,3-c]pyridinyl, furo[3,2-b]pyridinyl] or furo[2,3-b]pyridinyl), triazinylazepinyl, and the like.

The heterocyclyl ring may be optionally fused to an aryl or heteroaryl ring as defined herein provided the aryl and heteroaryl rings are monocyclic. Additionally, one or two ring carbon atoms in the heterocyclyl ring can optionally be replaced with a carbonyl group. When the heterocyclyl ring is partially saturated it can contain 1-3 ring double bonds provided that the ring is not aromatic.

When the term “substituted” is used with such “cycloalkyl”, “heterocyclyl” or “heteroaryl” groups, as in “substituted with 0 to 5 same or different substituents”, it should be understood that the cycloalkyl, heterocyclyl or heteroaryl may be substituted with the groups selected from those recited above and hereinafter as appropriate. Exemplary substituents include, but are not limited to, alkyl, substituted alkyl, halogen, nitro, cyano, and those groups recited above.

The terms “alkoxy”, “alkyloxy”, “alkenyloxy” and “alkynyloxy” refer to the groups of the structure OR and R′OR, wherein R and R′ is independently selected from the alkyl, alkenyl and alkynyl groups defined and recited above and hereinafter as appropriate.

The terms “alkylamino” or “dialkylamino” refer to an amino group wherein one or both of the hydrogen atoms are replaced with a group selected from the alkyl, alkenyl, alkynyl and cycloalkyl groups defined and recited above and hereinafter as appropriate.

The term “alkylthio” refers to the —SR″ radical, wherein R″ is selected from alkyl, alkenyl, alkynyl and cycloalkyl groups defined and recited above and hereinafter as appropriate.

The term “alkylsulfonyl” refers to a —SO₂R″ radical where R″ is selected from alkyl, alkenyl, alkynyl and cycloalkyl groups defined and recited above and hereinafter as appropriate. Exemplary alkylsulfonyl groups are methylsulfonyl, ethylsulfonyl, and the like.

The term “alkylsulfinyl” refers to a —S(═O)R″ radical where R″ is selected from alkyl, alkenyl, alkynyl and cycloalkyl groups defined and recited above and hereinafter as appropriate.

The term “alkoxycarbonyl” refers to a —C(═O)OR″ radical where R″ is selected from alkyl, alkenyl, alkynyl and cycloalkyl groups defined and recited above and hereinafter as appropriate. Exemplary alkoxycarbonyl groups are methoxycarbonyl, ethoxycarbonyl, and the like.

The term “halo” refers to fluoro, chloro, bromo, or iodo radicals. The terms “haloalkyl”, “haloalkenyl”, “haloalkynyl” and “halocycloalkyl” refers to alkyl, alkenyl, alkynyl and cycloalkyl radicals as defined above, substituted with one or more halogen atoms, including those substituted with different halogens. Exemplary groups are chloromethyl, trifluoromethyl, perfluoropropyl, trichloroethylenyl, chloroacetylenyl, and the like.

In a further embodiment of the present invention, the polymeric composition comprises a blend of a polymer and one or more compounds of Formula (I):

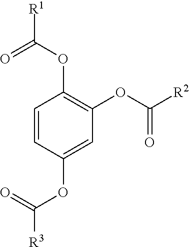

wherein R¹, R² and R³ independently represent C₁-C₁₂ alkyl; C₂-C₁₂ alkenyl; C₂-C₁₂ alkynyl; C₃-C₇ cycloalkyl; C₃-C₇ cycloalkenyl; C₃-C₇ cycloalkyl-C₁-C₁₂ alkyl; C₁-C₁₂ haloalkyl; C₂-C₁₂ haloalkenyl; C₂-C₁₂ haloalkynyl; C₃-C₇ halocycloalkyl; C₃-C₇ halocycloalkyl-C₁-C₁₂ alkyl; aryl-C₁-C₁₂ alkyl; aryl-C₂-C₁₂ alkenyl; aryl-C₂-C₁₂ alkynyl and aryl;
wherein aryl is substituted with 0 to 5 same or different substituents selected from a halogen atom; a hydroxyl group; C₁-C₁₈ alkyl; C₃-C₇ cycloalkyl; C₃-C₇ cycloalkyl-C₁-C₁₈ alkyl; C₂-C₁₈ alkenyl; C₂-C₁₈ alkynyl; C₁-C₁₈ haloalkyl; C₂-C₁₈ haloalkenyl; C₂-C₁₈ haloalkynyl; C₃-C₇ halocycloalkyl; C₃-C₇ halocycloalkyl-C₁-C₁₈ alkyl; C₁-C₁₈ alkoxy; C₃-C₇ cycloalkyloxy; C₂-C₁₈ alkenyloxy; C₂-C₁₈ alkynyloxy; C₁-C₁₈ alkylcarbonyloxy; C₁-C₁₈ haloalkoxy; a C₁-C₁₈ alkylthio; a C₁-C₁₈ alkylsulfinyl; a C₁-C₁₈ alkylsulfonyl; a C₁-C₁₈ haloalkylthio; a C₁-C₁₈ haloalkylsulfinyl; a C₁-C₁₈ haloalkylsulfonyl; an amino group; C₁-C₁₈ alkylcarbonylamino; mono(C₁-C₁₈ alkyl)amino; di-(C₁-C₁₈ alkyl)amino; hydroxy-C₁-C₁₈ alkyl; C₁-C₁₈ alkoxy-C₁-C₁₈ alkyl; C₁-C₁₈ alkylthio-C₁-C₁₈ alkyl; C₁-C₁₈ alkylsulfinyl-C₁-C₁₈ alkyl; C₁-C₁₈ alkylsulfonyl-C₁-C₁₈ alkyl; C₁-C₁₈ haloalkylthio-C₁-C₁₈ alkyl; C₁-C₁₈ haloalkylsulfinyl-C₁-C₁₈ alkyl; C₁-C₁₈ haloalkylsulfonyl-C₁-C₁₈ alkyl; cyano-C₁-C₁₈ alkyl; C₁-C₁₈ alkoxy-C₁-C₁₈ alkoxy; C₃-C₇ cycloalkyl-C₁-C₁₈ alkyloxy; C₁-C₁₈ haloalkoxy-C₁-C₁₈ alkoxy; cyano-C₁-C₁₈ alkoxy; C₁-C₁₈ acyl; C₁-C₁₈ alkoxyimino-C₁-C₁₈ alkyl; a carboxylic acid group; C₁-C₁₈ alkoxycarbonyl; carbamoyl; nitro; cyano; aryl; sulfonyl, sulfinyl, formyl, thiol, isocyanate or isothiocyanate mono(C₁-C₁₈ alkyl)aminocarbonyl; di-(C₁-C₁₈ alkyl)amino-carbonyl; and C₃-C₇ alkylene group formed with two adjacent substituent groups, wherein 1 to 3 carbon atoms in the alkylene group may be substituted with an atom selected from a group consisting of an oxygen atom, a sulfur atom, a nitrogen atom, and a carbon atom constituting a carbonyl.

In a yet further embodiment of the present invention, the polymeric composition comprises a blend of a polymer and one or more compounds of Formula (I):

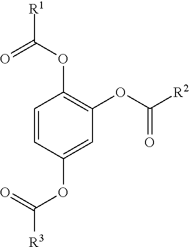

wherein R¹, R² and R³ independently represent C₁-C₁₂ alkyl.

In another embodiment of the present invention, the compounds of Formula (I) are prepared in a one-step esterification reaction between one equivalent of 1,2,4-benzenetriol (compound of Formula (III)) and at least 3.5 times excess of a carboxylic acid of Formula (II), as shown below

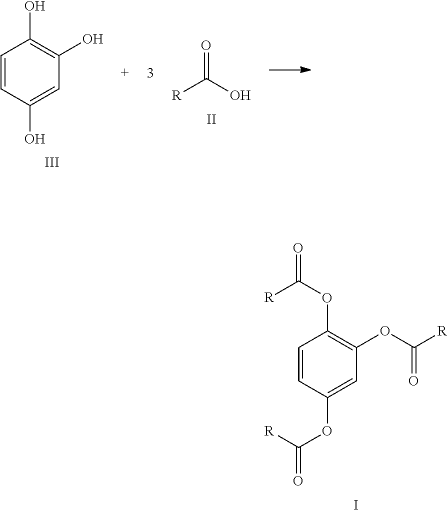

Use of the compound of Formula (I) as a plasticiser in a polymeric composition, such as PVC composition, is a further embodiment of the present invention.

Low molecular weight plasticisers are easy to use. They mix well with the polymer chains and the high density of chain-ends increases the free volume in the material. As the molecular weight of the plasticiser increases, there are more entanglements and less chain ends per mass of plasticiser. This changes many of the material properties: the glass transition temperature is raised, the tensile strength is increased, the tensile elongation is reduced, and the material usually becomes more difficult to process than a material plasticised with a low molecular weight plasticiser.

In the same way as lower molecular weight compounds induce a greater free volume in the material, the higher chain-end density of a branched polymer will provide more mobility in a blend than a linear polymer of the same molecular weight. Bulky low molecular weight plasticisers have also been shown to reduce the strength, stiffness and toughness of the material more efficiently than a plasticiser that is easily oriented along the polymer chains

Some plasticisers, which are compatible with PVC, are less likely to migrate from the PVC products, due to their molecular dimensions. PVC is basically rigid at ambient temperatures. This is due to the short distances between the molecular chains stabilised by Van-der-Waals inter-molecular interactions. When heated, the interatomic vibrational energy surpasses the energy of the Van-der-Waals interactions between the chains of PVC, thereby weakening them and increasing the molecular distances between them, resulting in softening of the polymer. When plasticisers are added to PVC at this stage, the plasticiser molecules can spatially arrange themselves between the PVC chains and prevent the polymeric chains from coming closer with each other. Upon cooling, the PVC chains cannot approach each other anymore and hence, kept apart by the plasticiser molecules even at ambient temperatures. Thus, softness of PVC is provided. This is the role of the plasticisers, and such process is technically called plasticising.

However, not many plasticisers are fully compatible with PVC from the molecular spatial and electronic point of view. PVC molecules have a weak dipole moment, which makes them slightly polarised and act like momentary dipoles induced by London forces. An ideal plasticiser must spatially and electronically match the PVC molecules in order to form the strongest possible interactions with the polymeric chains and prevent exudation from them. Therefore, the desired plasticiser molecule should not only have a relatively low molecular weight and a structure of linear alkenes or planar aromatic molecules, but also have such weak dipole moments. As a result, the PVC polymer molecules and the plasticiser molecules are electronically attracted to each other by induced dipole-dipole London forces and consequently stabilised by Van-der-Waals interactions. This will make the PVC softer and prevent the plasticiser from the exudation.

It was surprisingly found that the compounds of Formula (I) can be successfully used as plasticisers in the polymeric and PVC compositions. As explained above, they conform well to PVC from the molecular spatial and electronic point of view, keeping the required softness at minimal quantity, which results in high plasticising efficiency, and not easily migrating into air or water due to the strong Van-der-Waals interactions they form with the polymeric PVC chains.

In a further embodiment of the invention, the PVC composition contains about 10 to 1000 parts of plasticiser of Formula (I) per each 100 parts of the PVC polymer (PHR), preferably 20 to 200 PHR of the plasticiser. In a yet further embodiment, the PVC composition contains a mixture of different plasticisers of Formula (I).

In another embodiment of the present invention, the polymeric composition contains one or more stabilisers such as salts of the long-chain fatty acids, organic phosphites, epoxy compounds, tin stabilisers, and mixtures thereof. The stabilisers provide protection against the deficient PVC homopolymerisation and copolymerisation, and retard or even eliminate the process of polymer degradation. The total amount of the stabiliser present in the compositions ranges from 0.05 to 20 PHR, preferably from 0.2 to 10 PHR.

In a specific embodiment of the present invention, the stabiliser is a mixture of salts of long-chain fatty acids, such as stearates, oleates, laurelates and octoates (defined in the literatures as soaps), and epoxy compounds or organic phosphites. Exemplary soaps are calcium stearate, barium stearate, zinc stearate, cadmium stearate, calcium oleate, barium ricinolate, calcium laurelate, zinc octoate and their mixtures. Commercially available stabilisers are manufactured for example by Azko Interstab (calcium stearate/zinc stearate, CZ-19A, and barium stearate/cadmium stearate, BC-103L, mixtures), and by Bearlocher (barium stearate/zinc stearate mixture, UBZ-791).

Exemplary epoxy compound stabilizers are epoxy soybean oil such as Srapex 6.8, ESO, epoxy linseed oil, epoxy polybutadiene, epoxy methylstearate, epoxy stearate, epoxy ethylhexyl stearate, epoxy stearyl stearate, epoxy propyl isocyanalate 3-(2-case INO)-1,2-epoxy propane, bis-phenol A diglycidyl ether, vinyl dicyclohexanediepoxide, 2,2-bis-(4-hydroxyphenol) propane, and mixtures thereof. Organic phosphite stabilisers include diphenyldecyl phosphite, triphenyl phosphite, tris-nonylphenyl phosphite, tri-steareal phosphite, octyldiphenyl phosphite, and mixtures thereof. Tin stabilizers include tin dilaurate, dibutyl tin maleate, organic tin mercaptide and organic tin sulfonic amide, and mixtures thereof.

The above stabilisers may be used individually or in any combination. In addition, organic phosphites may be used in conjunction with the zinc stearate, barium stearate, cadmium stearate, and epoxy compound mixtures. The compositions of the present invention may include additional additives, such as anti-static agents, anti-fogging agents, ultra-violet inhibitors, anti-oxidants, light stabilisers, fire retardants, pigments, and mixtures thereof. These additives are generally known in the art and may be present in the compositions in an amount of 0.05-20 PHR, which is sufficient to impart the desired properties to the polymeric composition. Exemplary anti-static and anti-fogging agents are sorbitan fatty acid ester, sorbitol fatty acid ester, and glycerine fatty acid ester.

The polymeric compositions of the present invention are prepared by the methods known in the prior art, for example mixing the liquid ingredients together using a high or low intensity mixer, and addition of the solid PVC powder and other solid ingredients over a short time period while the mixing is continued, thereby forming a dispersion, which is degased under vacuum. The obtained crude mass then may be fused under a number of different heat conditions such as knife coated to a certain thickness and fused in an oven at varying temperature, in heated molds, in cold molds that are subsequently heated, and similar methods. Once fused, physical properties of the obtained plastic may be determined.

Dry compositions of the present invention may be prepared by combining the solid ingredients and mixing them in a Henschel mixer. The liquid ingredients then are slowly added. The mixture is blended until a temperature of about 88° C., which is below the PVC fusion temperature, is reached. This procedure produced a free-flowing powder of PVC particles having the other ingredients absorbed therein. The free-flowing powder then may be fused on a two-roll mill at a temperature of about 140° to 150° C. to produce a fused PVC sheet.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A polymeric composition comprising a blend of a polymer and one or more compounds of Formula (I): 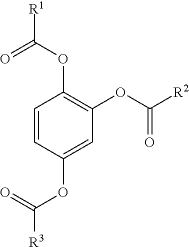
wherein R¹, R² and R³ independently represents C₁-C₁₈ alkyl; C₂-C₁₈ alkenyl; C₂-C₁₈ alkynyl; C₃-C₇ cycloalkyl; C₃-C₇ cycloalkenyl; C₃-C₇ cycloalkyl-C₁-C₁₈ alkyl; C₁-C₁₈ haloalkyl; C₂-C₁₈ haloalkenyl; C₂-C₁₈ haloalkynyl; C₃-C₇ halocycloalkyl; C₃-C₇ halocycloalkyl-C₁-C₁₈ alkyl; amino-C₁-C₁₈ alkyl; nitro-C₁-C₁₈ alkyl; C₁-C₁₈ alkylamino-C₁-C₁₈ alkyl; di-(C₁-C₁₈ alkyl)amino-C₁-C₁₈ alkyl; C₁-C₁₈ alkylthio-C₁-C₁₈ alkyl; C₁-C₁₈ alkylsulfinyl-C₁-C₁₈ alkyl; C₁-C₁₈ alkylsulfonyl-C₁-C₁₈ alkyl; C₁-C₁₈ haloalkylthio-C₁-C₁₈ alkyl; C₁-C₁₈ haloalkylsulfinyl-C₁-C₁₈ alkyl; C₁-C₁₈ haloalkylsulfonyl-C₁-C₁₈ alkyl; C₁-C₁₈ alkoxy-C₁-C₁₈ alkyl; hydroxy-C₁-C₁₈ alkyl; aryl-C₁-C₁₈ alkoxy-C₁-C₁₈ alkyl; C₂-C₂₄ polyethylene oxide; C₃-C₇ cycloalkyloxy-C₁-C₁₈ alkyl; C₃-C₇ cycloalkyl-C₁-C₁₈ alkyloxy-C₁-C₁₈ alkyl; aryloxy-C₁-C₁₈ alkyl; arylthio-C₁-C₁₈ alkyl; arylsulfinyl-C₁-C₁₈ alkyl; arylsulfonyl-C₁-C₁₈ alkyl; C₁-C₁₈ haloalkoxy-C₁-C₁₈ alkyl; aryl-C₁-C₁₈ alkyl; aryl-C₂-C₁₈ alkenyl; aryl-C₂-C₁₈ alkynyl; C₁-C₁₈ alkoxyimino-C₁-C₁₈ alkyl; aryloxyimino-C₁-C₁₈ alkyl; di-(C₁-C₁₈ alkoxy)C₁-C₁₈ alkyl; (R⁴R⁵N—C═O)C₁-C₁₈ alkyl; C₁-C₁₈ alkoxycarbonyl C₁-C₁₈ alkyl; C₁-C₁₈ alkylcarbonyl-C₁-C₁₈ alkyl; C₁-C₁₈ alkyl-carbonyloxy-C₁-C₁₈ alkyl; C₁-C₁₈ alkylidene aminooxy-C₁-C₁₈ alkyl; formyl-C₁-C₁₈ alkyl; C₁-C₁₈ alkylthio-C₁-C₁₈ alkoxy-C₁-C₁₈ alkyl; C₁-C₁₈ alkylsulfinyl-C₁-C₁₈ alkoxy-C₁-C₁₈ alkyl; C₁-C₁₈ alkylsulfonyl-C₁-C₁₈ alkoxy-C₁-C₁₈ alkyl; cyano-C₁-C₁₈ alkyl; cyano-C₁-C₁₈ alkoxy-C₁-C₁₈ alkyl; C₂-C₁₈ alkylidene amino; di-(C₁-C₁₈ alkyl)amino-C₁-C₁₈ alkylidene amino; NR⁴R⁵; aryl; heterocyclyl comprising 1 to 10 carbon atoms and one or more same or different heteroatoms selected from oxygen, sulfur optionally oxidized to sulfoxide or sulfone, and nitrogen; C₁-C₁₈ alkyl group substituted with heterocyclyl comprising 1 to 10 carbon atoms and one or more same or different heteroatoms selected from oxygen, sulfur optionally oxidized to sulfoxide or sulfone, and nitrogen; C₁-C₁₈ alkoxy-C₁-C₁₈ alkyl group substituted with heterocyclyl comprising 1 to 10 carbon atoms and one or more same or different heteroatoms selected from oxygen, sulfur optionally oxidized to sulfoxide or sulfone, and nitrogen;
R⁴ and R⁵ each independently represent a hydrogen atom; C₁-C₁₈ alkyl; aryl; C₁-C₁₈ alkoxy-C₁-C₁₈ alkyl; C₁-C₁₈ alkylcarbonyl; C₁-C₁₈ alkylthio carbonyl; C₁-C₁₈ alkoxycarbonyl; C₁-C₁₈ haloalkyl; C₃-C₇ cycloalkyl; C₃-C₇ cycloalkyl-C₁-C₁₈ alkyl; C₁-C₁₈ alkylsulfonyl; and arylsulfonyl;
R⁴ and R⁵ may be joined together to form a 5- to 6-membered ring with adjacent nitrogen atom, and the one or more carbon atoms in the ring may be substituted with a sulfur atom and an oxygen atom;
wherein aryl and heterocyclyl are substituted with 0 to 5 same or different substituents selected from a halogen atom; a hydroxyl group; C₁-C₁₈ alkyl; C₃-C₇ cycloalkyl; C₃-C₇ cycloalkyl-C₁-C₁₈ alkyl; C₂-C₁₈ alkenyl; C₂-C₁₈ alkynyl; C₁-C₁₈ haloalkyl; C₂-C₁₈ haloalkenyl; C₂-C₁₈ haloalkynyl; C₃-C₇ halocycloalkyl; C₃-C₇ halocycloalkyl-C₁-C₁₈ alkyl; C₁-C₁₈ alkoxy; C₃-C₇ cycloalkyloxy; C₂-C₁₈ alkenyloxy; C₂-C₁₈ alkynyloxy; C₁-C₁₈ alkylcarbonyloxy; C₁-C₁₈ haloalkoxy; a C₁-C₁₈ alkylthio; a C₁-C₁₈ alkylsulfinyl; a C₁-C₁₈ alkylsulfonyl; a C₁-C₁₈ haloalkylthio; a C₁-C₁₈ haloalkylsulfinyl; a C₁-C₁₈ haloalkylsulfonyl; an amino group; C₁-C₁₈ alkylcarbonylamino; mono(C₁-C₁₈ alkyl)amino; di-(C₁-C₁₈ alkyl)amino; hydroxy-C₁-C₁₈ alkyl; C₁-C₁₈ alkoxy-C₁-C₁₈ alkyl; C₁-C₁₈ alkylthio-C₁-C₁₈ alkyl; C₁-C₁₈ alkylsulfinyl-C₁-C₁₈ alkyl; C₁-C₁₈ alkylsulfonyl-C₁-C₁₈ alkyl; C₁-C₁₈ haloalkylthio-C₁-C₁₈ alkyl; C₁-C₁₈ haloalkylsulfinyl-C₁-C₁₈ alkyl; C₁-C₁₈ haloalkylsulfonyl-C₁-C₁₈ alkyl; cyano-C₁-C₁₈ alkyl; C₁-C₁₈ alkoxy-C₁-C₁₈ alkoxy; C₃-C₇ cycloalkyl-C₁-C₁₈ alkyloxy; C₁-C₁₈ haloalkoxy-C₁-C₁₈ alkoxy; cyano-C₁-C₁₈ alkoxy; C₁-C₁₈ acyl; C₁-C₁₈ alkoxyimino-C₁-C₁₈ alkyl; a carboxylic acid group; C₁-C₁₈ alkoxycarbonyl; carbamoyl; nitro; cyano; aryl; sulfonyl, sulfinyl, formyl, thiol, isocyanate or isothiocyanate mono(C₁-C₁₈ alkyl)aminocarbonyl; di-(C₁-C₁₈ alkyl)amino-carbonyl; and C₃-C₇ alkylene group formed with two adjacent substituent groups, wherein 1 to 3 carbon atoms in the alkylene group may be substituted with an atom selected from a group consisting of an oxygen atom, a sulfur atom, a nitrogen atom, and a carbon atom constituting a carbonyl.

2. The polymeric composition according to claim 1 comprising a blend of a polymer and one or more compounds of Formula (I): 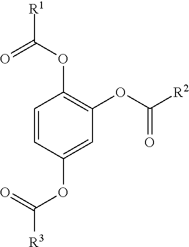
wherein R¹, R² and R³ independently represents C₁-C₁₂ alkyl; C₂-C₁₂ alkenyl; C₂-C₁₂ alkynyl; C₃-C₇ cycloalkyl; C₃-C₇ cycloalkenyl; C₃-C₇ cycloalkyl-C₁-C₁₂ alkyl; C₁-C₁₂ haloalkyl; C₂-C₁₂ haloalkenyl; C₂-C₁₂ haloalkynyl; C₃-C₇ halocycloalkyl; C₃-C₇ halocycloalkyl-C₁-C₁₂ alkyl; aryl-C₁-C₁₂ alkyl; aryl-C₂-C₁₂ alkenyl; aryl-C₂-C₁₂ alkynyl and aryl;
wherein aryl is substituted with 0 to 5 same or different substituents selected from a halogen atom; a hydroxyl group; C₁-C₁₈ alkyl; C₃-C₇ cycloalkyl; C₃-C₇ cycloalkyl-C₁-C₁₈ alkyl; C₂-C₁₈ alkenyl; C₂-C₁₈ alkynyl; C₁-C₁₈ haloalkyl; C₂-C₁₈ haloalkenyl; C₂-C₁₈ haloalkynyl; C₃-C₇ halocycloalkyl; C₃-C₇ halocycloalkyl-C₁-C₁₈ alkyl; C₁-C₁₈ alkoxy; C₃-C₇ cycloalkyloxy; C₂-C₁₈ alkenyloxy; C₂-C₁₈ alkynyloxy; C₁-C₁₈ alkylcarbonyloxy; C₁-C₁₈ haloalkoxy; a C₁-C₁₈ alkylthio; a C₁-C₁₈ alkylsulfinyl; a C₁-C₁₈ alkylsulfonyl; a C₁-C₁₈ haloalkylthio; a C₁-C₁₈ haloalkylsulfinyl; a C₁-C₁₈ haloalkylsulfonyl; an amino group; C₁-C₁₈ alkylcarbonylamino; mono(C₁-C₁₈ alkyl)amino; di-(C₁-C₁₈ alkyl)amino; hydroxy-C₁-C₁₈ alkyl; C₁-C₁₈ alkoxy-C₁-C₁₈ alkyl; C₁-C₁₈ alkylthio-C₁-C₁₈ alkyl; C₁-C₁₈ alkylsulfinyl-C₁-C₁₈ alkyl; C₁-C₁₈ alkylsulfonyl-C₁-C₁₈ alkyl; C₁-C₁₈ haloalkylthio-C₁-C₁₈ alkyl; C₁-C₁₈ haloalkylsulfinyl-C₁-C₁₈ alkyl; C₁-C₁₈ haloalkylsulfonyl-C₁-C₁₈ alkyl; cyano-C₁-C₁₈ alkyl; C₁-C₁₈ alkoxy-C₁-C₁₈ alkoxy; C₃-C₇ cycloalkyl-C₁-C₁₈ alkyloxy; C₁-C₁₈ haloalkoxy-C₁-C₁₈ alkoxy; cyano-C₁-C₁₈ alkoxy; C₁-C₁₈ acyl; C₁-C₁₈ alkoxyimino-C₁-C₁₈ alkyl; a carboxylic acid group; C₁-C₁₈ alkoxycarbonyl; carbamoyl; nitro; cyano; aryl; sulfonyl, sulfinyl, formyl, thiol, isocyanate or isothiocyanate mono(C₁-C₁₈ alkyl)aminocarbonyl; di-(C₁-C₁₈ alkylamino-carbonyl; and C₃-C₇ alkylene group formed with two adjacent substituent groups, wherein 1 to 3 carbon atoms in the alkylene group may be substituted with an atom selected from a group consisting of an oxygen atom, a sulfur atom, a nitrogen atom, and a carbon atom constituting a carbonyl.

3. The polymeric composition according to claim 2 comprising a blend of a polymer and one or more compounds of Formula (I): 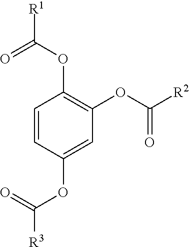
wherein R¹, R² and R³ independently represents C₁-C₁₂ alkyl.

4. The polymeric composition according claim 1, wherein said polymer is polyvinyl chloride (PVC).

5. The polymeric composition according to claim 1, wherein said composition contains 10 to 1000 PHR of the compound of Formula (I).

6. The polymeric composition according to claim 5, wherein said composition contains 20 to 200 PHR of the compound of Formula (I).

7. The polymeric composition according to claim 1, wherein said composition contains a mixture of compounds of Formula (I).

8. The polymeric composition according to claim 1, wherein said composition further contains one or more stabilisers.

9. The polymeric composition according to claim 8, wherein said stabilisers are selected from the group of salts of the long-chain fatty acids, organic phosphites, epoxy compounds, tin stabilisers, and mixtures thereof.

10. The polymeric composition according to claim 9, wherein said stabilisers are selected from the group of stearates, oleates, laurelates, octoates, and mixtures thereof.

11. The polymeric composition according to claim 8, wherein said stabilisers are added in an amount of 0.05-20 PHR.

12. The polymeric composition according to claim 11, wherein said stabilisers are added in an amount of 0.2 to 10 PHR.

13. The polymeric composition according to claim 1, wherein said composition further contains additives.

14. The polymeric composition according to claim 13, wherein said additives are selected from the group of anti-static agents, anti-fogging agents, ultra-violet inhibitors, anti-oxidants, light stabilisers, fire retardants, pigments, and mixtures thereof.

15. The polymeric composition according to claim 13, wherein said additives are added in an amount of 0.05-20 PHR.

16. A compound of Formula (I): 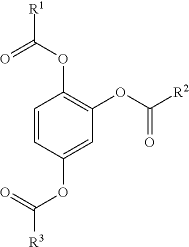
for use as a plasticiser in a polymeric composition;
wherein R¹, R² and R³ independently represents C₁-C₁₈ alkyl; C₂-C₁₈ alkenyl; C₂-C₁₈ alkynyl; C₃-C₇ cycloalkyl; C₃-C₇ cycloalkenyl; C₃-C₇ cycloalkyl-C₁-C₁₈ alkyl; C₁-C₁₈ haloalkyl; C₂-C₁₈ haloalkenyl; C₂-C₁₈ haloalkynyl; C₃-C₇ halocycloalkyl; C₃-C₇ halocycloalkyl-C₁-C₁₈ alkyl; amino-C₁-C₁₈ alkyl; nitro-C₁-C₁₈ alkyl; C₁-C₁₈ alkylamino-C₁-C₁₈ alkyl; di-(C₁-C₁₈ alkyl)amino-C₁-C₁₈ alkyl; C₁-C₁₈ alkylthio-C₁-C₁₈ alkyl; C₁-C₁₈ alkylsulfinyl-C₁-C₁₈ alkyl; C₁-C₁₈ alkylsulfonyl-C₁-C₁₈ alkyl; C₁-C₁₈ haloalkylthio-C₁-C₁₈ alkyl; C₁-C₁₈ haloalkylsulfinyl-C₁-C₁₈ alkyl; C₁-C₁₈ haloalkylsulfonyl-C₁-C₁₈ alkyl; C₁-C₁₈ alkoxy-C₁-C₁₈ alkyl; hydroxy-C₁-C₁₈ alkyl; aryl-C₁-C₁₈ alkoxy-C₁-C₁₈ alkyl; C₂-C₂₄ polyethylene oxide; C₃-C₇ cycloalkyloxy-C₁-C₁₈ alkyl; C₃-C₇ cycloalkyl-C₁-C₁₈ alkyloxy-C₁-C₁₈ alkyl; aryloxy-C₁-C₁₈ alkyl; arylthio-C₁-C₁₈ alkyl; arylsulfinyl-C₁-C₁₈ alkyl; arylsulfonyl-C₁-C₁₈ alkyl; C₁-C₁₈ haloalkoxy-C₁-C₁₈ alkyl; aryl-C₁-C₁₈ alkyl; aryl-C₂-C₁₈ alkenyl; aryl-C₂-C₁₈ alkynyl; C₁-C₁₈ alkoxyimino-C₁-C₁₈ alkyl; aryloxyimino-C₁-C₁₈ alkyl; di-(C₁-C₁₈ alkoxy)C₁-C₁₈ alkyl; (R⁴R⁵N-C=O)C₁-C₁₈ alkyl; C₁-C₁₈ alkoxycarbonyl C₁-C₁₈ alkyl; C₁-C₁₈ alkylcarbonyl-C₁-C₁₈ alkyl; C₁-C₁₈ alkyl-carbonyloxy-C₁-C₁₈ alkyl; C₁-C₁₈ alkylidene aminooxy-C₁-C₁₈ alkyl; formyl-C₁-C₁₈ alkyl; C₁-C₁₈ alkylthio-C₁-C₁₈ alkoxy-C₁-C₁₈ alkyl; C₁-C₁₈ alkylsulfinyl-C₁-C₁₈ alkoxy-C₁-C₁₈ alkyl; C₁-C₁₈ alkylsulfonyl-C₁-C₁₈ alkoxy-C₁-C₁₈ alkyl; cyano-C₁-C₁₈ alkyl; cyano-C₁-C₁₈ alkoxy-C₁-C₁₈ alkyl; C₂-C₁₈ alkylidene amino; di-(C₁-C₁₈ alkyl)amino-C₁-C₁₈ alkylidene amino; NR⁴R⁵; aryl; heterocyclyl comprising 1 to 10 carbon atoms and one or more same or different heteroatoms selected from oxygen, sulfur optionally oxidized to sulfoxide or sulfone, and nitrogen; C₁-C₁₈ alkyl group substituted with heterocyclyl comprising 1 to 10 carbon atoms and one or more same or different heteroatoms selected from oxygen, sulfur optionally oxidized to sulfoxide or sulfone, and nitrogen; C₁-C₁₈ alkoxy-C₁-C₁₈ alkyl group substituted with heterocyclyl comprising 1 to 10 carbon atoms and one or more same or different heteroatoms selected from oxygen, sulfur optionally oxidized to sulfoxide or sulfone, and nitrogen;
R⁴ and R⁵ each independently represent a hydrogen atom; C₁-C₁₈ alkyl; aryl; C₁-C₁₈ alkoxy-C₁-C₁₈ alkyl; C₁-C₁₈ alkylcarbonyl; C₁-C₁₈ alkylthio carbonyl; C₁-C₁₈ alkoxycarbonyl; C₁-C₁₈ haloalkyl; C₃-C₇ cycloalkyl; C₃-C₇ cycloalkyl-C₁-C₁₈ alkyl; C₁-C₁₈ alkylsulfonyl; and arylsulfonyl;
R⁴ and R⁵ may be joined together to form a 5- to 6-membered ring with adjacent nitrogen atom, and the one or more carbon atoms in the ring may be substituted with a sulfur atom and an oxygen atom;
wherein aryl and heterocyclyl are substituted with 0 to 5 same or different substituents selected from a halogen atom; a hydroxyl group; C₁-C₁₈ alkyl; C₃-C₇ cycloalkyl; C₃-C₇ cycloalkyl-C₁-C₁₈ alkyl; C₂-C₁₈ alkenyl; C₂-C₁₈ alkynyl; C₁-C₁₈ haloalkyl; C₂-C₁₈ haloalkenyl; C₂-C₁₈ haloalkynyl; C₃-C₇ halocycloalkyl; C₃-C₇ halocycloalkyl-C₁-C₁₈ alkyl; C₁-C₁₈ alkoxy; C₃-C₇ cycloalkyloxy; C₂-C₁₈ alkenyloxy; C₂-C₁₈ alkynyloxy; C₁-C₁₈ alkylcarbonyloxy; C₁-C₁₈ haloalkoxy; a C₁-C₁₈ alkylthio; a C₁-C₁₈ alkylsulfinyl; a C₁-C₁₈ alkylsulfonyl; a C₁-C₁₈ haloalkylthio; a C₁-C₁₈ haloalkylsulfinyl; a C₁-C₁₈ haloalkylsulfonyl; an amino group; C₁-C₁₈ alkylcarbonylamino; mono(C₁-C₁₈ alkyl)amino; di-(C₁-C₁₈ alkyl)amino; hydroxy-C₁-C₁₈ alkyl; C₁-C₁₈ alkoxy-C₁-C₁₈ alkyl; C₁-C₁₈ alkylthio-C₁-C₁₈ alkyl; C₁-C₁₈ alkylsulfinyl-C₁-C₁₈ alkyl; C₁-C₁₈ alkylsulfonyl-C₁-C₁₈ alkyl; C₁-C₁₈ haloalkylthio-C₁-C₁₈ alkyl; C₁-C₁₈ haloalkylsulfinyl-C₁-C₁₈ alkyl; C₁-C₁₈ haloalkylsulfonyl-C₁-C₁₈ alkyl; cyano-C₁-C₁₈ alkyl; C₁-C₁₈ alkoxy-C₁-C₁₈ alkoxy; C₃-C₇ cycloalkyl-C₁-C₁₈ alkyloxy; C₁-C₁₈ haloalkoxy-C₁-C₁₈ alkoxy; cyano-C₁-C₁₈ alkoxy; C₁-C₁₈ acyl; C₁-C₁₈ alkoxyimino-C₁-C₁₈ alkyl; a carboxylic acid group; C₁-C₁₈ alkoxycarbonyl; carbamoyl; nitro; cyano; aryl; sulfonyl, sulfinyl, formyl, thiol, isocyanate or isothiocyanate mono(C₁-C₁₈ alkyl)aminocarbonyl; di-(C₁-C₁₈ alkyl)amino-carbonyl; and C₃-C₇ alkylene group formed with two adjacent substituent groups, wherein 1 to 3 carbon atoms in the alkylene group may be substituted with an atom selected from a group consisting of an oxygen atom, a sulfur atom, a nitrogen atom, and a carbon atom constituting a carbonyl.

17. The compound according to claim 16, wherein said polymer is PVC.

18. A process for manufacturing the compound of Formula (I) 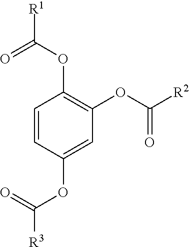
comprising the step of esterification of one equivalent of 1,2,4-benzenetriol and at least 3.5 equivalents of the carboxylic acid of Formula (II): 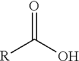
wherein R, R¹, R², and R³ independently represent C₁-C₁₈ alkyl; C₂-C₁₈ alkenyl; C₂-C₁₈ alkynyl; C₃-C₇ cycloalkyl; C₃-C₇ cycloalkenyl; C₃-C₇ cycloalkyl-C₁-C₁₈ alkyl; C₁-C₁₈ haloalkyl; C₂-C₁₈ haloalkenyl; C₂-C₁₈ haloalkynyl; C₃-C₇ halocycloalkyl; C₃-C₇ halocycloalkyl-C₁-C₁₈ alkyl; amino-C₁-C₁₈ alkyl; nitro-C₁-C₁₈ alkyl; C₁-C₁₈ alkylamino-C₁-C₁₈ alkyl; di-(C₁-C₁₈ alkylamino-C₁-C₁₈ alkyl; C₁-C₁₈ alkylthio-C₁-C₁₈ alkyl; C₁-C₁₈ alkylsulfinyl-C₁-C₁₈ alkyl; C₁-C₁₈ alkylsulfonyl-C₁-C₁₈ alkyl; C₁-C₁₈ haloalkylthio-C₁-C₁₈ alkyl; C₁-C₁₈ haloalkylsulfinyl-C₁-C₁₈ alkyl; C₁-C₁₈ haloalkylsulfonyl-C₁-C₁₈ alkyl; C₁-C₁₈ alkoxy-C₁-C₁₈ alkyl; hydroxy-C₁-C₁₈ alkyl; aryl-C₁-C₁₈ alkoxy-C₁-C₁₈ alkyl; C₁-C₂₄ polyethylene oxide; C₃-C₇ cycloalkyloxy-C₁-C₁₈ alkyl; C₃-C₇ cycloalkyl-C₁-C₁₈ alkyloxy-C₁-C₁₈ alkyl; aryloxy-C₁-C₁₈ alkyl; arylthio-C₁-C₁₈ alkyl; arylsulfinyl-C₁-C₁₈ alkyl; arylsulfonyl-C₁-C₁₈ alkyl; C₁-C₁₈ haloalkoxy-C₁-C₁₈ alkyl; aryl-C₁-C₁₈ alkyl; aryl-C₂-C₁₈ alkenyl; aryl-C₂-C₁₈ alkynyl; C₁-C₁₈ alkoxyimino-C₁-C₁₈ alkyl; aryloxyimino-C₁-C₁₈ alkyl; di-(C₁-C₁₈ alkoxy)C₁-C₁₈ alkyl; (R⁴R⁵N—C═O)C₁-C₁₈ alkyl; C₁-C₁₈ alkoxycarbonyl C₁-C₁₈ alkyl; C₁-C₁₈ alkylcarbonyl-C₁-C₁₈ alkyl; C₁-C₁₈ alkyl-carbonyloxy-C₁-C₁₈ alkyl; C₁-C₁₈ alkylidene aminooxy-C₁-C₁₈ alkyl; formyl-C₁-C₁₈ alkyl; C₁-C₁₈ alkylthio-C₁-C₁₈ alkoxy-C₁-C₁₈ alkyl; C₁-C₁₈ alkylsulfinyl-C₁-C₁₈ alkoxy-C₁-C₁₈ alkyl; C₁-C₁₈ alkylsulfonyl-C₁-C₁₈ alkoxy-C₁-C₁₈ alkyl; cyano-C₁-C₁₈ alkyl; cyano-C₁-C₁₈ alkoxy-C₁-C₁₈ alkyl; C₂-C₁₈ alkylidene amino; di-(C₁-C₁₈ alkyl)amino-C₁-C₁₈ alkylidene amino; NR⁴R⁵; aryl; heterocyclyl comprising 1 to 10 carbon atoms and one or more same or different heteroatoms selected from oxygen, sulfur optionally oxidized to sulfoxide or sulfone, and nitrogen; C₁-C₁₈ alkyl group substituted with heterocyclyl comprising 1 to 10 carbon atoms and one or more same or different heteroatoms selected from oxygen, sulfur optionally oxidized to sulfoxide or sulfone, and nitrogen; C₁-C₁₈ alkoxy-C₁-C₁₈ alkyl group substituted with heterocyclyl comprising 1 to 10 carbon atoms and one or more same or different heteroatoms selected from oxygen, sulfur optionally oxidized to sulfoxide or sulfone, and nitrogen;
R⁴ and R⁵ each independently represent a hydrogen atom; C₁-C₁₈ alkyl; aryl; C₁-C₁₈ alkoxy-C₁-C₁₈ alkyl; C₁-C₁₈ alkylcarbonyl; C₁-C₁₈ alkylthio carbonyl; C₁-C₁₈ alkoxycarbonyl; C₁-C₁₈ haloalkyl; C₃-C₇ cycloalkyl; C₃-C₇ cycloalkyl-C₁-C₁₈ alkyl; C₁-C₁₈ alkylsulfonyl; and arylsulfonyl;
R⁴ and R⁵ may be joined together to form a 5- to 6-membered ring with adjacent nitrogen atom, and the one or more carbon atoms in the ring may be substituted with a sulfur atom and an oxygen atom;
wherein aryl and heterocyclyl are substituted with 0 to 5 same or different substituents selected from a halogen atom; a hydroxyl group; C₁-C₁₈ alkyl; C₃-C₇ cycloalkyl; C₃-C₇ cycloalkyl-C₁-C₁₈ alkyl; C₂-C₁₈ alkenyl; C₂-C₁₈ alkynyl; C₁-C₁₈ haloalkyl; C₂-C₁₈ haloalkenyl; C₂-C₁₈ haloalkynyl; C₃-C₇ halocycloalkyl; C₃-C₇ halocycloalkyl-C₁-C₁₈ alkyl; C₁-C₁₈ alkoxy; C₃-C₇ cycloalkyloxy; C₂-C₁₈ alkenyloxy; C₂-C₁₈ alkynyloxy; C₁-C₁₈ alkylcarbonyloxy; C₁-C₁₈ haloalkoxy; a C₁-C₁₈ alkylthio; a C₁-C₁₈ alkylsulfinyl; a C₁-C₁₈ alkylsulfonyl; a C₁-C₁₈ haloalkylthio; a C₁-C₁₈ haloalkylsulfinyl; a C₁-C₁₈ haloalkylsulfonyl; an amino group; C₁-C₁₈ alkylcarbonylamino; mono(C₁-C₁₈ alkyl)amino; di-(C₁-C₁₈ alkyl)amino; hydroxy-C₁-C₁₈ alkyl; C₁-C₁₈ alkoxy-C₁-C₁₈ alkyl; C₁-C₁₈ alkylthio-C₁-C₁₈ alkyl; C₁-C₁₈ alkylsulfinyl-C₁-C₁₈ alkyl; C₁-C₁₈ alkylsulfonyl-C₁-C₁₈ alkyl; C₁-C₁₈ haloalkylthio-C₁-C₁₈ alkyl; C₁-C₁₈ haloalkylsulfinyl-C₁-C₁₈ alkyl; C₁-C₁₈ haloalkylsulfonyl-C₁-C₁₈ alkyl; cyano-C₁-C₁₈ alkyl; C₁-C₁₈ alkoxy-C₁-C₁₈ alkoxy; C₃-C₇ cycloalkyl-C₁-C₁₈ alkyloxy; C₁-C₁₈ haloalkoxy-C₁-C₁₈ alkoxy; cyano-C₁-C₁₈ alkoxy; C₁-C₁₈ acyl; C₁-C₁₈ alkoxyimino-C₁-C₁₈ alkyl; a carboxylic acid group; C₁-C₁₈ alkoxycarbonyl; carbamoyl; nitro; cyano; aryl; sulfonyl, sulfinyl, formyl, thiol, isocyanate or isothiocyanate mono(C₁-C₁₈ alkyl)aminocarbonyl; di-(C₁-C₁₈ alkyl)amino-carbonyl; and C₃-C₇ alkylene group formed with two adjacent substituent groups, wherein 1 to 3 carbon atoms in the alkylene group may be substituted with an atom selected from a group consisting of an oxygen atom, a sulfur atom, a nitrogen atom, and a carbon atom constituting a carbonyl.

19. The process according to claim 18, wherein said process comprises the step of esterification of one equivalent of 1,2,4-benzenetriol and at least 3.5 equivalents of the carboxylic acid of Formula (II), 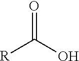
wherein R, R¹, R², and R³ are identical and represent C₁-C₁₂ alkyl; C₂-C₁₂ alkenyl; C₂-C₁₂ alkynyl; C₃-C₇ cycloalkyl; C₃-C₇ cycloalkenyl; C₃-C₇ cycloalkyl-C₁-C₁₂ alkyl; C₁-C₁₂ haloalkyl; C₂-C₁₂ haloalkenyl; C₂-C₁₂ haloalkynyl; C₃-C₇ halocycloalkyl; C₃-C₇ halocycloalkyl-C₁-C₁₂ alkyl; aryl-C₁-C₁₂ alkyl; aryl-C₂-C₁₂ alkenyl; aryl-C₂-C₁₂ alkynyl and aryl;
wherein aryl is substituted with 0 to 5 same or different substituents selected from a halogen atom; a hydroxyl group; C₁-C₁₈ alkyl; C₃-C₇ cycloalkyl; C₃-C₇ cycloalkyl-C₁-C₁₈ alkyl; C₂-C₁₈ alkenyl; C₂-C₁₈ alkynyl; C₁-C₁₈ haloalkyl; C₂-C₁₈ haloalkenyl; C₂-C₁₈ haloalkynyl; C₃-C₇ halocycloalkyl; C₃-C₇ halocycloalkyl-C₁-C₁₈ alkyl; C₁-C₁₈ alkoxy; C₃-C₇ cycloalkyloxy; C₂-C₁₈ alkenyloxy; C₂-C₁₈ alkynyloxy; C₁-C₁₈ alkylcarbonyloxy; C₁-C₁₈ haloalkoxy; a C₁-C₁₈ alkylthio; a C₁-C₁₈ alkylsulfinyl; a C₁-C₁₈ alkylsulfonyl; a C₁-C₁₈ haloalkylthio; a C₁-C₁₈ haloalkylsulfinyl; a C₁-C₁₈ haloalkylsulfonyl; an amino group; C₁-C₁₈ alkylcarbonylamino; mono(C₁-C₁₈ alkyl)amino; di-(C₁-C₁₈ alkyl)amino; hydroxy-C₁-C₁₈ alkyl; C₁-C₁₈ alkoxy-C₁-C₁₈ alkyl; C₁-C₁₈ alkylthio-C₁-C₁₈ alkyl; C₁-C₁₈ alkylsulfinyl-C₁-C₁₈ alkyl; C₁-C₁₈ alkylsulfonyl-C₁-C₁₈ alkyl; C₁-C₁₈ haloalkylthio-C₁-C₁₈ alkyl; C₁-C₁₈ haloalkylsulfinyl-C₁-C₁₈ alkyl; C₁-C₁₈ haloalkylsulfonyl-C₁-C₁₈ alkyl; cyano-C₁-C₁₈ alkyl; C₁-C₁₈ alkoxy-C₁-C₁₈ alkoxy; C₃-C₇ cycloalkyl-C₁-C₁₈ alkyloxy; C₁-C₁₈ haloalkoxy-C₁-C₁₈ alkoxy; cyano-C₁-C₁₈ alkoxy; C₁-C₁₈ acyl; C₁-C₁₈ alkoxyimino-C₁-C₁₈ alkyl; a carboxylic acid group; C₁-C₁₈ alkoxycarbonyl; carbamoyl; nitro; cyano; aryl; sulfonyl, sulfinyl, formyl, thiol, isocyanate or isothiocyanate mono(C₁-C₁₈ alkyl)aminocarbonyl; di-(C₁-C₁₈ alkyl)amino-carbonyl; and C₃-C₇ alkylene group formed with two adjacent substituent groups, wherein 1 to 3 carbon atoms in the alkylene group may be substituted with an atom selected from a group consisting of an oxygen atom, a sulfur atom, a nitrogen atom, and a carbon atom constituting a carbonyl.

20. The process according to claim 19, wherein said process comprises the step of esterification of one equivalent of 1,2,4-benzenetriol and at least 3.5 equivalents of the carboxylic acid of Formula (II), 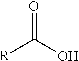
wherein R represents C₁-C₁₂ alkyl.
